(19) United States
(12) Patent Application Publication
(10) Pub. No.: US 2024/0189249 A1
(43) Pub. Date: Jun. 13, 2024

(54) CONDUCTIVE STIMULI-RESPONSIVE COLLOIDAL MICROGELS AND THEIR FILM HOMOLOGUES: SYNTHESIS USING CATECHOL GROUPS AS CROSSLINKING AND DOPING AGENTS, AND MECHANO-ELECTRICAL PROPERTIES

(71) Applicants: UNIVERSITE DE PAU ET DES PAYS DE L'ADOUR, Pau (FR); CENTRE NATIONAL DE LA RECHERCHE SCIENTIFIQUE - CNRS -, Paris (FR)

(72) Inventors: Garbine AGUIRRE UGARTE, Billere (FR); Pierre MARCASUZAA, Lons (FR); Laurent BILLON, Saint Faust (FR)

(21) Appl. No.: 18/552,472

(22) PCT Filed: Mar. 24, 2022
(86) PCT No.: PCT/EP2022/057837

§ 371 Date: Sep. 26, 2023

(30) Foreign Application Priority Data
Mar. 26, 2021 (EP) 21305379.6

(51) Int. Cl. A61K 9/70; A61K 8/02; A61K 8/11; A61K 8/81; A61K 9/50; A61K 45/06; A61Q 1/00; A61Q 19/00

(52) U.S. Cl. CPC A61K 9/7007; A61K 8/0204; A61K 8/11; A61K 8/8158; A61K 9/5026; A61K 9/5089; A61K 45/06; A61Q 1/00; A61Q 19/00; A61K 2800/805

(57) ABSTRACT

The present invention relates to a composition comprising an oligo(ethylene glycol)-based polymers microgel crosslinked with a catechol cross-linker, said microgel comprises microgel particles, and wherein the microgel particles have a shell of conductive polymer. This composition is able to form cohesive films spontaneously with enhanced conductive and mechano-electrical properties.

### FIELD OF THE INVENTION

### Background of the Invention

The primary challenge of topical treatment is the penetration of therapeutic compounds through the primary skin barrier known as stratum corneum. In the last years, several techniques and formulations have been developed with the aim of overcoming skin barriers and reaching skin malignancies by favoring the therapeutic compounds penetration into the deeper layers of the skin.

In this regard, among different approaches developed to increase skin permeability, iontophoresis is a relevant technique. This technique is based on the application of an electric field with a low electrical potential difference between the skin and the delivery systems to enhance the delivery across the skin through preexisting pores of the stratum corneum or through new pores. In this scenario, materials able to intrinsically generate an electric filed by soft and slight mechanical deformations are very interesting being able to break the skin barrier while delivering therapeutic molecules due to mechanical deformations. In the last years, several authors have attempted to accurately study the mechano-electrical properties of soft ionic macroscopic hydrogels.

A first generation of new biocompatible and multi-responsive oligo(ethylene glycol)-based microgels have been synthesized. Said microgel can form Self-Assembled Microgel Films (SAMF) presenting mechano-electrical behavior. Regarding the mechano-electrical properties of SAMF (hybrid or not), an output voltage of 25 mV has been observed by compressing those films (WO2016/110615).

Moreover, it has been demonstrated the opportunity to encapsulate high amounts of active molecules together with inorganic nanoparticles (Boularas et al. 2015, Macromol. Rapid Commun., 36:79) by such colloidal system enhancing their potential applications as delivery systems. The effective encapsulation of active molecules has been also observed in the case of Self-Assembled Microgel Films (SAMF).

However, the mechano-electrical properties of these microgels and SAMF are insufficient for delivering active principles and favoring their penetration into the deeper layers of the skin.

As the generation of an electric field by soft mechanical deformation is a requirement to break the skin barrier, it is hence necessary to enhance electrical property of microgels and to develop microgels able to form cohesive films spontaneously with conductive and mechano-electrical properties in order to deliver therapeutic or cosmetic compounds through the skin barrier.

### SUMMARY OF THE INVENTION

The inventors have synthetized conductive stimuli-responsive microgels, able to form cohesive films spontaneously with enhanced conductive and mechano-electrical properties.

Thus, the present invention relates to a composition comprising an oligo(ethylene glycol)-based polymers microgel crosslinked with a catechol cross-linker, said microgel comprises microgel particles, and wherein the microgel particles have a shell of conductive polymer.

The invention also concerns a process of preparing a composition as described above, said process comprising the step of preparing a microgel, said microgel comprising particles, via precipitation polymerization of monomers, wherein said monomers are crosslinked with a catechol cross-linker, and preparing a conductive shell via polymerization of a conductive polymer on the microgel particles.

The microgels according to the invention are capable of self-assembling in order to form a film consisting of one or more layers of microgels, by a process of drying or evaporating an aqueous suspension of said microgels.

Hence, in another aspect, the present invention relates to a Self-Assembled Microgel Film obtained by solvent evaporation, and to a process of obtaining a Self-Assembled Microgel Films comprising a step of applying on keratin materials a composition according to the invention.

Advantageously, the films formed according to the present invention generate an electric potential via compression effect. Output voltages generated after finger compression for microgel self-assembled films according to the invention were around 150 mV and even superior to 200 mV in some cases. The output voltage is also maintained constant at least 1 minute.

Advantageously, when using a series of films or Self-Assembled Microgel Films, a high electrical potential can be generated being possible to amplify it combining the appropriate number of films in series.

Hence, in an embodiment, the present invention also relates to a series of films or Self-Assembled Microgel Films wherein the film or Self-Assembled Microgel Films are connected.

From the point of view of cosmetic and therapeutic applications, reversible pore induction in cell membranes and lipid bilayer membranes has been observed at 150-250 mV (several seconds) (H. Inada, A.-H. Ghanem, W. I. Higure, Pharm. Res., 1994, 11, 687-697). Therefore, the films formed according the present invention should be able to create new pores in the skin enhancing the penetration of the active molecules.

Hence, the present invention also relates to a cosmetic product and to a make-up or a skin care method comprising a step of applying on keratinous materials such a cosmetic product.

In a last aspect, the present invention concerns a therapeutic product, its use in therapy and its use for delivering active agents through the stratum corneum.

### FIGURES

FIG. 1 represents evolutions of partial conversions of M2 (a), M3 (b) microgels.

FIG. 2 represents average hydrodynamic diameters as a function of pH at 25° C.

FIG. 3 represents average hydrodynamic diameters as a function of temperature at pH 6 (a) and pH 3 (b) of microgels synthesized.

FIG. 4 represents AFM images in dried state of M1 (a) and M2 (b) and immersed in alkaline solution at pH of M2 (c) microgels.

FIG. 5 represents TEM micrographs in dried state of M1 (a) and M2 (b) microgels.

FIG. 6 represents AFM micrographs of the surface of self-assembled M2 purified microgel films. Non-colored area (a), colored area (b,c).

FIG. 7 represents AFM images in dried state of microgels synthesized in height (a) and adhesion (b) modes.

FIG. 8 represents transmission electron micrographs in dried state of microgels synthesized.

FIG. 9 represents STEM-EDS micrographs in dried state of M2-PEDOT25 microgel.

FIG. 10 represents potential difference (mV) as a function of compression length (%) of M2 self-assembled film.

FIG. 11 represents output voltage (mV) generated after constant finger compression for microgel self-assembled films formed using catechol (a) and PSS as dopant (b).

FIG. 12 represents STEM-EDS cross section image of self-assembled slightly cross-linked core-shell microgels with 10 wt % (a) and 25 wt % of PEDOT (b) film.

FIG. 13 represents output voltage generated as a function of force applied.

FIG. 14 represents effect of films area on output voltage for slightly cross-linked self-assembled microgel with 10 wt % of PEDOT.

FIG. 15 represents effect of films thickness on output voltage for slightly cross-linked self-assembled microgel with 10 wt % of PEDOT.

FIG. 16 represents a schematic illustration of the films placed in series (a). Output voltage (mV) generated after constant finger compression for self-assembled microgel films with 350 μm and 750 μm thickness (b).

### DETAILED DESCRIPTION OF THE INVENTION

In a first aspect, the present invention concerns a composition comprising an oligo(ethylene glycol)-based polymers microgel crosslinked with a catechol cross-linker, said microgel comprises microgel particles, and wherein the microgel particles have a shell of conductive polymer.

### Microgel

“Microgel” in the sense of the invention are compositions in the form of an aqueous dispersion of microgel particles or in the form of a film comprising microgel particles, wherein the microgel particles are crosslinked polymer in the form of particles having a size that varies from 100 nm to 500 nm in the dry state (i.e. containing less than 2% by weight of water), preferably between 125 and 450 nm, preferably between 150 and 250 nm, more preferably of the order of 200 nm. Typically, the particles are spherical.

It must be noted that microgel is distinct from a hydrogel. Hydrogel is a bulky material chemically formed without any possibilities to re-shaped it. Microgel is particles in colloidal state dispersed in water media. Such colloidal solution can be in-situ shaped by drying the solution without any chemistry reaction but only physical-chemical interactions.

In an embodiment, microgels are oligo(ethylene glycol)-based polymers microgel.

In an embodiment, microgels are obtainable by aqueous phase precipitation polymerization of the following three monomers:
di(ethylene glycol) methyl ether methacrylate (MeO₂MA);
an oligo(ethylene glycol) methyl ether methacrylate (M(EO)ₙMA), n being an integer ranging from 3 to 12, preferably ranging from 8 to 10;
a monomer of formula CR₁R₂═CR₃R₄ in which R₁, R₂, R₃ and R₄ represent a hydrogen, a halogen or a hydrocarbon group, at least one of the four groups comprising a —COOH or —COO— M+ group, M+ representing a cation,
   crosslinked with a catechol cross-linker.

MeO₂MA represents for example 50 mol % to 90 mol % of the total number of moles of the monomers, M(EO)ₙMA preferably represents 10 to 50 mol % of the total number of moles of the monomers and the monomer of formula CR₁R₂═CR₃R₄ preferably represents 0.1 mol % to 20 mol % of the total number of moles of the monomers, the sum of these three contents being equal to 100%.

The molar ratio between MeO₂MA and M(EO)ₙMA is preferably between 1:1 and 20:1, for example between 5:1 and 10:1.

According to one embodiment, MeO₂MA represents for example 80 to 90 mol % of the total number of moles of the three monomers, M(EO)ₙMA preferably represents 5 to 15 mol % of the total number of moles of the monomers and the monomer of formula CR₁R₂═CR₃R₄ preferably represents 0.1 to 10 mol % of the total number of moles of the monomers, the sum of these three contents being equal to 100%.

In an embodiment, M(EO)ₙMA is preferably an oligo(ethylene glycol) methyl ether methacrylate also denoted OEGMA.

The monomer of formula CR₁R₂═CR₃R₄ is preferably such that R₁ and R₂ each represent a hydrogen, R₃ represents H or an alkyl group, preferably a C1-C6 alkyl group, optionally substituted with —OH or —COOH, and R₄ represents, independently of R₃, the —COOH group or an alkyl group, preferably a C1-C6 alkyl group, optionally substituted with —OH or —COOH. The alkyl group may be methyl, ethyl or n-butyl. According to one particular embodiment. R₁ and R₂ each represent a hydrogen and R₃ and R₄ independently represent —H, —COOH, or COOH.

The monomer of formula CR₁R₂═CR₃R₄ may for example be chosen from methyl acrylic, methyl methacrylic, ethyl acrylic, ethyl methacrylic, n-butyl acrylic and n-butyl methacrylic acids, vinylic monomer comprising a carboxylic group.

According to one embodiment, the monomer of formula CR₁R₂═CR₃R₄ may be methacrylic acid or itaconic acid.

Acrylic acid may be excluded from the definition of the monomer of formula CR₁R₂═CR₃R₄ in certain cases.

In a particular embodiment, microgels are obtainable by aqueous phase precipitation polymerization of di(ethylene glycol) methyl ether methacrylate (MeO₂MA), oligo(ethylene glycol) methyl ether methacrylate (OEGMA) and methacrylic acid (MAA), wherein these monomers are crosslinked with a catechol cross-linker.

In a yet particular embodiment, MeO₂MA represents for example 80 to 90 mol % of the total number of moles of the three monomers, OEGMA represents 5 to 15 mol % of the total number of moles of the monomers and methacrylic acid (MAA) preferably represents 0.1 to 10 mol % of the total number of moles of the monomers, the sum of these three contents being equal to 100%.

### Catechol Cross-Linker

According to the present invention, the catechol cross-linker is the sole cross-linker. The reactivity of catechols with radicals was applied to synthesize cross-linked microgels, without addition of any other cross-linker by Xue et al. (DOI: 10.1021/acs.macromol.7b01304, Macromolecules 2017, 50, 5285-5292).

Xue and coworkers reported that catechols can react with propagating radicals forming cross-linked structures. They proposed three possible mechanisms for the formation of cross-linking network structure during radical copolymerization of different monomers and dopamine-methacrylamide. These mechanisms are the covalent coupling of the catechol groups, the formation of hydrogen bonds between the hydroxyl groups of catechol, and the reaction between the catechol group of one polymer chain and the radical of another propagating chain.

With the aim of determining the type of mechanisms followed by catechols for cross-linking points, different experiments were carried out. Without wishing to be bound by any theory, the inventors considered the reaction between the catechol group of one polymer chain and the radical of another propagating chain as main mechanisms for cross-linking points formation between copolymer chains.

Hence and as used herein, “catechol cross-linker” refers to any molecule bearing a catechol group, allowing the synthesis of cross-linked microgels by polymerization, preferably by a reaction between the catechol group of one polymer chain and the radical of another propagating chain.

Advantageously, with catechol cross-linker, the microgel could be achieved by using aqueous phase precipitation polymerization without addition of any other cross-linker and surfactant stabilizer.

Advantageously also, the use of catechols group with cross-linker functionality, without the need of existing propagating radicals of polymer in the reaction medium to form microgel opens the possibility to synthetize microgels by one-step batch precipitation polymerization.

In an embodiment, the catechol cross-linker comprises an acrylamide or methacrylamide group.

In an embodiment, the catechol cross-linker is chosen among dopamine-acrylamide or dopamine methacrylamide and is preferably dopamine-acrylamide.

Both dopamine-acrylamide or dopamine methacrylamide bear unprotected catechol group allowing the synthesis of cross-linked microgels.

In an embodiment, the catechol cross-linker represents from 1 to 20 mol % of the total number of moles of the monomers.

Inner structure of the microgels can depend on the amount of cross-linker used. According to several embodiment, two different microstructures were obtained: highly cross-linked microgels using from 10 to 20 mol % of the total number of moles of the monomers of catechol cross-linker, preferably from 10 to 15 mol % of the total number of moles of the monomers of catechol cross-linker, and slightly cross-linked microgels using from 1 to 10 mol % of the total number of moles of the monomers of catechol cross-linker, preferably from 1 to 5 mol % of the total number of moles of the monomers of catechol cross-linker.

In a preferred embodiment, 10 mol % of the total number of moles of the monomers of catechol cross-linker is used.

### Conductive Polymer

As used herein, the term “conductive polymer” means a polymer or an oligomer that is inherently or intrinsically capable of electrical conductivity.

Preferably, the conductive polymer is a biocompatible conductive polymer. As used herein “biocompatible” shall mean any material that does not cause injury or death to the human or induce an adverse reaction in a human when placed in intimate contact with the human tissues.

In an embodiment, the conductive polymer is selected among poly(3,4-ethylenedioxythiophene) (PEDOT), PEDOT derivatives and poly(3-hexylthiophen) (P3HT).

As used herein “poly(3,4-ethylenedioxythiophene) (PEDOT)” means a polymer obtained by polymerization of EDOT (3,4-ethylenedioxythiophene) monomers. The terms “PEDOT derivatives” means a polymer obtained by polymerization of EDOT monomers derivatives selected among hydroxymethyl-EDOT, vinyl-EDOT, allyl ether-EDOT, COOH-EDOT, MeOH-EDOT, silane-EDOT, acrylate-EDOT, sulfonate-EDOT, amine-EDOT or a mixture thereof. The term “poly(3-hexylthiophen) (P3HT)” means a polymer obtained by polymerization of 3-hexylthiophen monomers.

In a preferred embodiment, the conductive polymer is PEDOT.

Hence the catechol group possess a double functionality and acts as a cross-linker and a dopant.

As used herein, the term “dopant” covers compounds which can significantly enhanced the conductivity of a conductive polymer by interaction between the dopant and the conductive polymer.

The use of catechol groups as dopant allows direct polymerization of EDOT onto microgels and offers an interesting and elegant alternative to conventional and more toxic dopants such as PSS.

Advantageously, the inventors demonstrate that catechol groups are more efficient as a dopant agent that PSS in the case of incorporating PEDOT shell onto microgel particles and allows the synthesis of a conductive shell using microgel particles as seed.

### Conductive Shell

As used herein, the term “conductive shell” or “conductive polymer shell” means a continuous or discontinuous deposit of conductive polymers at the surface of the microgel, said deposit of conductive polymer being physically (i.e. adsorbed) and/or chemically (i.e. grafted) linked to microparticles of microgel.

According to the present invention, the conductive shell of the microgel is synthesized using microgel particles as seed. Without wishing to be bound by any theory, it is hypothesized that conductive polymer polymerized at the surface of the microgel particles and forms domains onto microgel particles but without completely covering the surface of the microgel.

When microgels assemble in order to form a film, it is also hypothesized that these domains of conductive polymers form a percolated phase of conductive polymer.

Similar conductive values were obtained independently of the amount of PEDOT. The reason could be related with the location of PEDOT incorporated. In the case of using slightly cross-linked microgel as seed, PEDOT polymerized mainly in the surface of the microgels. On the other hand, in the case of the highly cross-linked microgel, due to the distribution of catechol groups into particles, some PEDOT was polymerized into the surface but also into microgel particles. Therefore, in both cases, the PEDOT polymerized at the surface could be similar leading to similar conductivity values.

Hence, in a preferred embodiment, the conductive shell is a discontinuous conductive shell meaning that conductive polymer polymerized at the surface of microparticles, forming domains onto the surface of particles.

Advantageously, even with their discontinuous conductive shell, microgels according to the invention can form cohesive and elastic film.

Thus, the inventors demonstrate the suitability of catechol cross-linker to efficiently adhere conductive polymer and preferably PEDOT, to the surface of microgel particles. This functionality of catechol cross-linker as dopant is better than the use of conventional poly(styrene sulfonate) (PSS) dopant in order to attach PEDOT to the surface of microgel particles.

Typically, the conductive shell is obtained by polymerization of EDOT (3,4-ethylenedioxythiophene) monomers or EDOT monomers derivatives selected among hydroxymethyl-EDOT, vinyl-EDOT, allyl ether-EDOT, COOH-EDOT, MeOH-EDOT, silane-EDOT, acrylate-EDOT, sulfonate-EDOT, amine-EDOT or a mixture thereof or 3-hexylthiophen monomers.

In a preferred embodiment, the monomer is EDOT.

### Process

In a further aspect, the present invention relates to process of preparing a composition, said process comprising the steps of:
Preparing a microgel via precipitation polymerization of monomers in the presence of a catechol cross-linker, wherein said microgel comprises particles and wherein the monomers are selected among:
   di(ethylene glycol) methyl ether methacrylate (MeO₂MA):
   an oligo(ethylene glycol) methyl ether methacrylate (M(EO)ₙMA), n being an integer ranging from 3 to 12, preferably ranging from 8 to 10,
   a monomer of formula CR₁R₂═CR₃R₄ in which R₁, R₂, R₃ and R₄ represent a hydrogen, a halogen or a hydrocarbon group, at least one of the four groups comprising a —COOH or —COO⁻M⁺ group, M⁺ representing a cation,
Preparing a conductive shell via polymerization on microgel particles.

According to the invention, the conductive shell is advantageously synthesized using the catechol groups of the cross-linker.

The monomers and the catechol cross-linker are those previously described herein before.

In an embodiment, the conductive shell is prepared via polymerization of at least one monomer selected among EDOT (3,4-ethylenedioxythiophene), EDOT derivatives and 3-hexylthiophen.

In an embodiment, the precipitation polymerization comprises a step of bringing into contact in an aqueous phase, in the presence of the catechol cross-linker the monomers described above, at a temperature comprises between 40° C. and 90° C., and preferably at a temperature of 70° C.

In an embodiment, in the step of preparing microgel, the polymerization of the monomers may be initiated by addition of a water-soluble radical initiator, for example potassium persulfate (KPS) at a temperature comprises between 40° C. and 90° C., and preferably at a temperature of 70° ° C.

In an embodiment, in the step of preparing conductive shell, the polymerization of the conductive monomers may be also initiated by addition of an oxidative agent, for example ammonium persulfate (APS).

In an embodiment, the step of preparing conductive shell is conducted at a temperature comprises between 20° C. and 70° C., and preferably at 40° C.

In an embodiment, the conductive monomer represents from 5 to 30% by weight, relative to the weight of the microgel, preferably from 7 to 27% by weight, relative to the weight of the microgel.

Films, Self-Assembled Microgel Films, process of obtaining Films and Self-Assembled Microgel Films and Series of Self-Assembled Microgel Films

The microgels according to the invention are capable of assembling in order to form a film consisting of one or more layers of microgels, by a process of drying or evaporating an aqueous suspension of said microgels.

The thickness of each layer ranges from 10 to 1000 microns, preferably from 100 to 800 microns, more preferably from 100 to 400 microns or from 400 to 800 microns.

In an embodiment, the thickness of the layer is around 350 microns.

In another embodiment, the thickness of the layer is around 750 microns.

In an embodiment, the thickness of the film can be increased through the deposition of different layers onto the electrode.

Several layers of the film can be deposed onto the electrode to obtain an increase of output voltage generated.

Hence, the film can have a thickness that varies, ranging in some embodiments from 10 microns to 5.0 millimeters, preferably from 350 microns to 4.0 millimeters, preferably from 700 microns to 3.0 millimeters.

In an embodiment, the film area is comprised between 1E-05 m² and 6E-05 m². In an embodiment. the films are prepared by a process of drying or evaporating solvent at a temperature comprised between 20° C. and 40° ° C., preferably at 35° C.

Typically, the films of microgel particles can be formed according to a step of placing an aqueous microgel dispersion prepared, for example according to the process described above into a mold, and a step of drying the water dispersion. Drying can be performed by placing the mold at a temperature higher than 30° C., preferably around 35° C. or being ambient temperature.

Microgel according to the invention are also capable of forming a cohesive and elastic film. It is not necessary in the context of the invention to encapsulate or support the microgels in order to form a film; consequently; interaction between the microgels and keratin materials on which they are formed after water evaporation of an aqueous dispersion of the microgel particles is optimal.

Hence, the invention also concerns a process of obtaining a Self-Assembled Microgel Films comprising a step of applying on keratin materials a composition according to the invention, wherein the said Self-Assembled Microgel Films is obtained by solvent evaporation of the composition.

Typically, the self-assembled microgel film is obtained by simple drying at ambient temperature.

Typically, keratin materials are selected among the skin, the scalp, the hair, the nail, the lips, the eyebrow. Preferably the composition according to the invention is applied onto the skin.

These conductive microgels of the invention may thus be used as film-forming agent in therapeutic or cosmetic compositions, so as to improve the hold of these compositions on keratin materials and enhance the penetration of the active agent through stratum corneum due to their mechanoelectrical properties.

### Series of Self-Assembled Microgel Films

In an embodiment, and with the aim of increasing the electrical potential generated, multiple films or Self-Assembled Microgel Films can be connected in series.

Hence, in an embodiment, the present invention also relates to a series of films or Self-Assembled Microgel Films wherein each film or Self-Assembled Microgel Film is connected respectively to another film or Self-Assembled Microgel Film.

In an embodiment, films or Self-Assembled Microgel Films are connected through an electrical cable or wire linking the bottom electrode of one film or Self-Assembled Microgel Film with the upper electrode of another one.

In an embodiment, 2 to 50 films or Self-Assembled Microgel Films are connected, preferably 2 to 10, more preferably, 2 to 6 films or Self-Assembled Microgel Films are connected.

Advantageously, when using a series of films or Self-Assembled Microgel Films, a high electrical potential can be generated being possible to amplify it combining the appropriate number of films in series.

### Mechanoelectrical Properties

The composition and the films formed according to the present invention generate an electric potential via compression effect.

Output voltages generated after finger compression for microgel self-assembled films according to the invention were around 150 mV and even superior to 200 mV in some cases. The output voltage is also maintained constant at least 1 minute.

From the point of view of cosmetic and therapeutic applications, reversible pore induction in cell membranes and lipid bilayer membranes has been observed at 150-250 mV (several seconds) (H. Inada, A.-H. Ghanem, W. I. Higure, Pharm. Res., 1994, 11, 687-697). Therefore, the films formed according the present invention should be able to create new pores in the skin enhancing the penetration of the active molecules.

These properties make it possible to envisage the use of the composition of the invention and of the film that they form for the preparation of cosmetic or pharmaceutical product. These products stimulate keratin materials, preferably the skin, in order to deliver cosmetic or therapeutic agent via compression effect. The cosmetic or therapeutic agent may be entrapped in microgels or be present in the product.

Active agent loaded microgel

In an embodiment, the microgels are loaded with an active agent. By “loaded” is meant that the microgel particles include an amount of an active agent(s). As such, an amount of active agent is present in the microgel particle and may be viewed as entrapped in the microgel particle. The term “entrap” means that the active agent is located within the polymer network of the microgel. The network of the crosslinked polymer can form a barrier around the active-agent that can be suppressed by some physical change in the network. The entrapped active agent may not be linked to the crosslinked polymer with a covalent bond. The entrapped active agent can have electrostatic interactions, Van der Walls bonds or hydrogen bonds with the crosslinked polymer, that can be engaged between C═C bonds of —OH groups of the organic molecules and ethylene glycol moieties of the crosslinked polymer.

Microgels according to the invention can advantageously entrap active agent and encapsulate high amounts of different molecules.

In an embodiment, active agent are hydrophobic molecules.

In an embodiment, the active agent can be a cosmetic agent or a therapeutic agent.

The “amount of the active agent in the loaded microgel” is the weight (in microgram (μg)) of the active agent that is entrapped in the crosslinked polymer per 1 mg of crosslinked polymer in the loaded microgel. The “amount of the active agent in the loaded microgel” is also mentioned as the “entrapped substance amount” in the rest of the description.

Preparation of active-agent loaded microgel has been described in patent application WO2019/07740. Briefly, active-agent loaded microgel can be prepared according to the steps of:
preparing a dispersion of unloaded microgel particles in water
preparing a feeding solution of the active agent;
mixing the microgel obtained and the solution of the active agent causing encapsulation of the active agent in the microgel particles; and
recovering active-agent loaded microgel particles.

Typically, unloaded microgel particles are prepared by a precipitation polymerization method as described above in the presence of a catechol cross-linker.

Mixing step of active substance solution and unloaded microgel dispersion preferably comprises a step of heating at a temperature that is higher than the volume phase transition temperature of the unloaded microgel particles, and a step of cooling the obtained dispersion of loaded microgels at ambient temperature (25° C.).

The feeding solution of the active agent can be obtained by dissolution of a determined amount of the active agent in an appropriate solvent. Complete dissolution of a determined amount of the active substance in the solvent can be performed at a temperature being from ambient temperature to a temperature that is above the volume phase transition temperature of the unloaded microgel particles.

The “amount of the active agent in the feeding solution” also called “the feeding substance amount” in the following description is the weight of the active agent in the feeding solution (in μg or mg) per 1 mg of unloaded microgel particles that are used to entrap the active substance. The feeding substance amount unit may be written in a shorter way “mg/mg” or “microgram/mg”.

As described in WO 2019/077404, this process enables a high Entrapment Efficiency EE %.

The Entrapment Efficiency (EE %) is defined as the ratio of the weight of the active agent that is entrapped in the loaded microgels and the amount of the active agent that is contained in the feeding solution. The Entrapment Efficiency (EE %) can also be defined as the ratio A/B of the entrapped substance amount (A) and the feeding substance amount (B), as defined in the present application.

Active agent can be encapsulated into microgels that are in the form of an aqueous dispersion, or into microgels that have been prepared in the form of a film according to the description above.

Typically, the process for the preparation of active-agent loaded microgel in the form of a film comprises the step of:
preparing a feeding solution of the active agent in a solvent,
preparing a film of unloaded microgel particles,
immersing the film in the feeding solution so as to cause swelling of the film and diffusion of the active substance into the film, and
recovering the microgels that can be in the form of an active substance loaded microgel film.

The films are prepared as described above.

The step of immersing the film can be performed at 25° C. for at least 12 hours or 24 hours.

### Cosmetic Product

In an embodiment, the present invention concerns a cosmetic product comprising a composition as described above and at least one cosmetic agent.

In an embodiment, the microgel particles entrap the cosmetic agent. Microgels then can be named “loaded microgels” or “loaded microgel particles”.

The cosmetic agent includes but is not limited to chemicals, compounds, small or large molecules, extracts, formulations or combinations that are known to induce or cause at least one effect on skin tissue.

The microgel of the composition according to the invention can be in the form of an aqueous dispersion or in the form of film or a series of films. The cosmetic composition can be in the form of a make-up product, a skin care product, a hair care product.

As long as the purpose and effect of the present invention are not impaired, cosmetic product of the present invention can further contain any acceptable excipients, in addition to the composition of the present invention.

### Make-Up or Skin Care Method

In an embodiment, the present invention also relates to a make-up or a skin care method comprising a step of applying on keratinous materials, a cosmetic product as described above, and applying a compression on said product.

In an embodiment, the make-up or skin care method comprises the following steps:
applying on keratinous materials a cosmetic product as described above, the microgel of the composition being in the form of an aqueous dispersion or a series of aqueous dispersion;
waiting in order to obtained by solvent evaporation a self-assembled microgel film;
eventually connecting self-assembled microgel films
applying a compression on the self-assembled microgel film obtained.

In an embodiment, the make-up or skin care method comprises the following steps:
applying on keratinous materials a cosmetic product as described above, the microgel of the composition being in the form of a film or a series of films:
eventually connecting the series of films
applying a compression on the film.

Output voltages generated after compression of the product comprising the conductive microgel according to the invention stimulate keratin materials and allow to deliver cosmetic material through stratum corneum into superficial layers of the skin.

Typically, the applied force is around 10 to 15 N being this value range similar to that applied with a finger during the common application of a cream.

All the features applying to the composition according to the invention and all the features applying to the cosmetic product that have been described before also apply to the make-up or skin care method.

### Therapeutic Product

In an embodiment, the present invention also related to a therapeutic product comprising a composition as described above and at least one therapeutic agent.

The phrase “therapeutic agent” “which is interchangeably referred to herein as “drug” or “active agent” or therapeutically active agent”, describes a compound which exhibits a beneficial pharmacological effect when administered to a subject and hence can be used in the treatment of a condition that benefits from this pharmacological effect.

As long as the purpose and effect of the present invention are not impaired, therapeutic product according the present invention contains at least one therapeutic agent and optionally any acceptable excipients, in addition to the composition of the present invention.

In an embodiment, the microgel particles entrap the therapeutic agent. Microgels then can be named “loaded microgels” or “loaded microgel particles”.

Output voltages generated after compression of the product comprising the conductive microgel according to the invention stimulate keratin materials and allow to deliver therapeutic agent through stratum corneum into superficial and deep layers of the skin in order to deliver the therapeutic agent.

A method for Delivering an Active Agent

In an embodiment, the present invention also concerns a method for delivering a therapeutic agent comprising a step of applying on keratin materials a therapeutic product as described above and applying a compression on said product.

In yet an embodiment, the present invention also concerns a method for delivering a cosmetic agent comprising a step of applying on keratin materials a cosmetic product as described above and applying a compression on said product.

In an embodiment, the method for delivering a therapeutic agent comprises the following steps:
applying on keratinous materials a therapeutic product as described above, the microgel of the composition being in the form of an aqueous dispersion or series of aqueous dispersion;
waiting in order to obtained by solvent evaporation a self-assembled microgel film;
eventually connecting self-assembled microgel films;
applying a compression on the self-assembled microgel film obtained.

In another embodiment, the method for delivering a therapeutic agent comprises the following steps:
applying on keratinous materials a therapeutic product as described above, the microgel of the composition being in the form of a film or a series of film;
eventually connecting the series of film;
applying a compression on the film.

In yet an embodiment, the present invention also related to a therapeutic agent for use in therapy, wherein said therapeutic agent is delivered via the composition according to the invention.

In an embodiment, the therapeutic agent for use in therapy is delivered via the following steps:
applying on keratinous materials a therapeutic product as described above, the microgel of the composition being in the form of an aqueous dispersion or series of aqueous dispersion;
waiting in order to obtained by solvent evaporation a self-assembled microgel film;
eventually connecting self-assembled microgel films applying a compression on the self-assembled microgel film obtained.

In yet another embodiment, the therapeutic agent for use in therapy is delivered via the following steps:
applying on keratinous materials a therapeutic product as described above, the microgel of the composition being in the form of a film or a series of film;
eventually connecting the series of film
applying a compression on the film.

Output voltages generated after compression of the product comprising the conductive microgel according to the invention stimulate keratin materials and allow to deliver therapeutic material through stratum corneum into superficial and deep layers of the skin.

Typically, the applied force is around 10 to 15 N.

All the features applying to the composition according to the invention and all the features applying to the therapeutic product that have been described before also apply to the method for delivering the therapeutic agent.

### EXAMPLES

In the following examples, the following reagents were used.

TABLE 1
Chemical structures, names and abbreviations of reagents used

| Chemical structure | Name | Abbreviation |
|---|---|---|
| 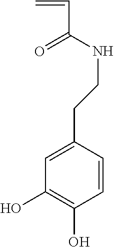 | Dopamine acrylamide | DA |
| 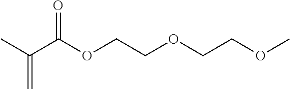 | Di(ethylene glycol) methyl ether methacrylate | MeO₂MA |
| 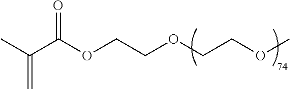 | Oligo(ethylene glycol) methyl ether methacrylate | OEGMA |
| 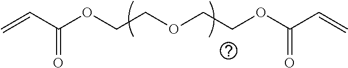 | Poly(ethylene glycol) diacrylate | OEGDA |
| 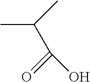 | Methacrylic acid | MAA |
| 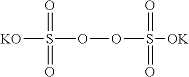 | Potassium persulfate | KPS |
| 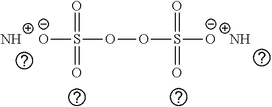 | Ammonium persulfate | APS |
| 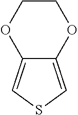 | 3-4-ethylenedioxythiophene | EDOT |
| 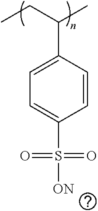 | Poly(sodium 4-styrenesulfonate) | PSS |

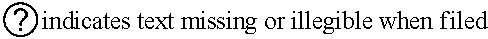

### Example 1:

### Synthesis of Microgels Using Dopamine-Acrylamide (DA) as Cross-Linker

Table 2 hereinafter shows the recipes and the reaction conditions used in the synthesis of microgel using DA as cross-linker.

TABLE 2
Recipes used to produce microgels using DA as cross-linker

| | [MeO₂MA]₀ | [OEGMA]₀ | [MAA]₀ | [DA]₀ | [KPS]₀ |
|---|---|---|---|---|---|
| | mmol · | mmol · | mmol · | mmol · | mmol · |
| Microgel | L⁻¹ | L⁻¹ | L⁻¹ | L⁻¹ | L⁻¹ |
| M1 | 83.9 | 9.36 | 4.99 | 1.92 | 0.86 |
| M2 | 83.9 | 9.36 | 4.99 | 11.52 | 0.86 |
| M3 | 83.9 | 9.36 | 4.99 | 14.40 | 0.86 |

Prior to microgel synthesis, DA was synthesized and characterized following the procedure described by Patil et al. (N. Patil, C. Falentin-Daudre, C. Jerome, C. Detrembleur, Polym. Chem., 2015, 6, 2919-2933).

Then, microgels were synthesized by precipitation polymerization in a 250 mL 3-neck round-bottom flask by following the procedure and recipe. Briefly, 5.14 mmol of MeO₂MA, 0.573 mmol of OEGMA, and 57.5 g of “Milli-Q” grade water were placed into a 250 mL 3-neck round-bottom flask. The reactor content was stirred at 150 rpm and purged with nitrogen for 45 min to remove oxygen at room temperature. Then, 0.305 mmol of MAA dissolved in 2 mL of “Milli-Q” grade water together with variable amounts of DA dissolved in 2 mL of ethanol were added to the jacketed glass reactor and the mixture was heated up to 70° C. After adding the initiator (14.3 mg of KPS dissolved in 2.5 mL of degassed water), the polymerization reaction was allowed to continue under nitrogen atmosphere while stirring for 6 h. Finally, the reaction mixture was subsequently cooled to 25° C. maintaining the stirring, and the final dispersion was purified by several centrifugation-redispersion cycles (10,000 rpm, 30 min) with “Milli-Q” grade water.

### 1. Kinetic Studies of the Precipitation Copolymerization

The evolution of the partial conversions of MeO₂MA, OEGMA, MAA and DA in the synthesis of microgel were determined by proton nuclear magnetic resonance spectroscopy (¹H NMR). For that, the surfactant free precipitation polymerization was performed as described above but adding trioxane as internal standard (10% mol M). ¹H NMR spectra were recorded at 400 MHz on a Bruker spectrometer, using D₂O/H₂O as solvent. The partial conversions of MeO₂MA, OEGMA, MAA, and DA were determined by the following expression:

$${x}_{M} = 1 - \frac{{I}_{M ( t )} / {I}_{Trioxane ( x )}}{{I}_{M ( 0 )} / {I}_{Trioxane ( 0 )}}$$

where I_{M} and I_{Trioxane} are the values of the peak integration of monomer M and internal standard (trioxane) at initial time t₀ and sampling time t.

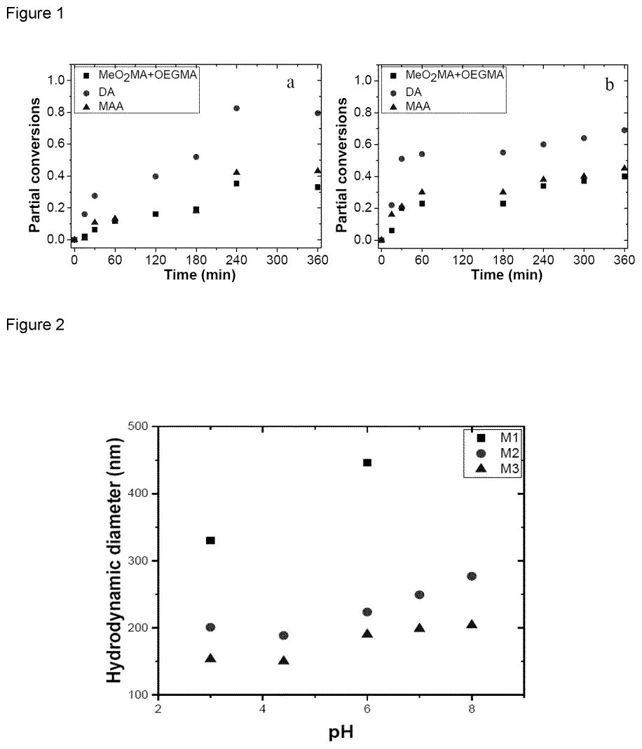

The final partial conversions of the monofunctional methacrylate monomers and the DA cross-linker are shown in FIG. 1. As can be seen, a limiting conversion of main monomers is observed reacting only the 40% of them. Without wishing to be bound by theory, the reason of this limiting conversion could be the scavenging effect of DA.

With the aim of confirming that DA molecules are able to react and incorporate into microgel particles, different measurements were carried out. Xue and coworkers reported that catechols can react with propagating radicals forming cross-linked structures. In this sense, they propose three possible mechanisms for the formation of cross-linking network structure during radical copolymerization of different monomers and dopamine-methacrylamide. These mechanisms are the covalent coupling of the catechol groups, the formation of hydrogen bonds between the hydroxyl groups of catechol, and the reaction between the catechol group of one polymer chain and the radical of another propagating chain.

With the aim of determining the type of mechanisms followed by catechols for cross-linking points. different experiments were carried out. In the case of covalent coupling of catechol groups, the characteristic absorbance with λmax˜280 nm shift to λmax˜267 nm. However, both microgels only exhibit one characteristic absorbance at 280 nm (results not disclosed), indicating the absence of covalent coupled catechols.

For hydrogen bonds formation between catechol groups determination, sodium borate (3.25 mM) was added to final microgel dispersions. It is known that borax can form cyclic bidentate p-benzenediol subunits with catechols which could provoke the disintegration/swelling of microgel particles. After 24 h of incubation, the maximum absorbance peak does not change and the swelling ratio of both microgels is close to 1 meaning that the inner structure of them is the same as the initial one.

Therefore, but without wishing to be bound by any theory, the inventors considered the reaction between the catechol group of one polymer chain and the radical of another propagating chain as main mechanisms for cross-linking points formation between copolymer chains s.

### 2. Swelling-to-Collapse Transition of Microgels

Colloidal characteristics of the microgels synthesized, such as the average hydrodynamic particle diameters at different temperatures and pHs, were measured by Photon Correlation Spectroscopy (PCS, Zetasizer Nano ZS instrument, Malvern Instruments). In all the measurements, the pH was controlled using different buffered media at an ionic strength of 1 mM.

In order to study the pH-sensitivity, measurements were carried out at 20° C. from pH 3 to pH 8 taking three measurements every pH unit at 1mg/mL sample concentration. The optimized stabilizing time of measurements was 5 min. As can be observed in FIG. 2, the pH-sensitivity of the microgels is the expected one being microgel particles collapsed below the VPTpH and swollen above it due to the ionization of carboxylic groups.

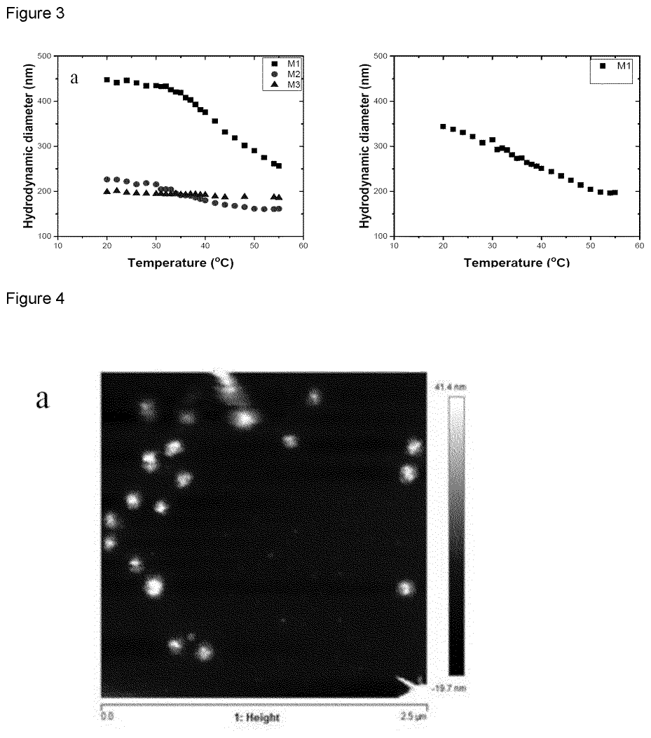

To study the thermal behavior. measurements were carried out at pH 6 from 20 to 55° C, taking measurements every 2° C. except from 30 to 40° C., that they were carried out per grade. As can be observed in FIG. 3, the thermal behavior of the microgels is the conventional one: particles are swollen by decreasing the temperature below VPTT (Volume Phase Transition Temperature) and they are collapsed at temperatures above VPTT. In the case of M1 microgel, at 55° C, the complete collapse of microgel particles was not achieved (see FIG. 3a). In order to enhance the collapse of MI microgel particles, the thermo-responsiveness was studied at pH 3 (see FIG. 3b). Interestingly, thanks to the small size of the microgel particles synthesized, they present color properties at wet conditions, but these are lost at dry state. In order to corroborate colored properties at wet state. film was formed in a UV-Vis cuvette and its absorbance was measured at dry and wet states (adding water to the cuvette). For film forming, some microgel dispersion was added to the cuvette wall. No difference is observed between wet and dry states in terms of absorbance i.e. the absorbance peak that appears is related to the characteristic peak of catechol groups. Maybe the drying process was not the suitable one to order microgel particles and present color properties.

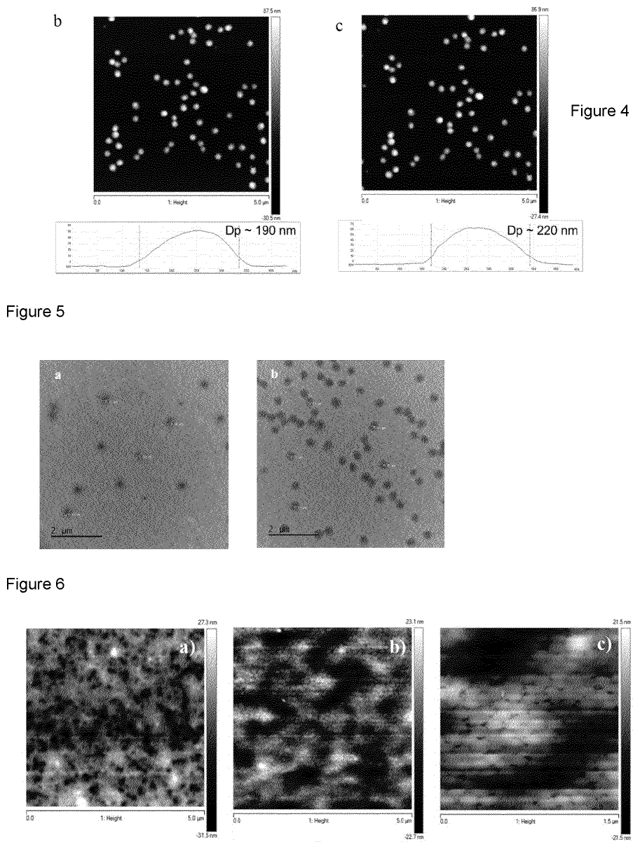

Images by Atomic Force Microscopy (AFM) of microgel particles at both dried and hydrated states were recorded on a Multi mode 8 (BrukerNano). For that, a droplet of microgel dispersion (10⁻³ wt %) was deposited on a clean wafer silicon and left to dry under ambient conditions prior to AFM surface mapping of the surface on air and immerged in alkaline solution (pH8). FIG. 4 shows the surface mapping and cross-sectional analysis of M1 and M2 microgels in dry and hydrated states by addition of alkaline solution at pH 8. Monodisperse and spherical particles are observed, in the case of M2 microgel (FIGS. 4b and 4c). By contrast, in the case of M1 the shape of microgel particles is not very well defined (FIG. 4a). This could be due to the low cross-linking density of the particles.

In addition, after the addition of alkaline solution, the size of the microgel particles is larger due to the electrostatic repulsions of anionic methacrylate units. However, the height of the swollen particles is smaller than the width and this could be because the attractive forces between the substrate and the microgel particles avoiding the complete swelling of the microgel (see Table 3).

TABLE 3
Values of microgel particles width, height and width/height
ratio in dried and hydrated states.

| | Dry state | | | Hydrated state (pH 8) | | |
|---|---|---|---|---|---|---|
| | Width | Heigth | Width/ | Width | Heigth | Width/ |
| Microgel | (nm) | (nm) | Heigth | (nm) | (nm) | Heigth |
| M1 | 190 | 60 | 3.2 | 220 | 70 | 3.2 |
| M2 | 110 | 40 | 2.8 | — | — | — |

### 3. Direct Observation by TEM

M1 and M2 microgel samples were sent to Bordeaux Imaging Center (BIC) for transmission electron microscopy (TEM) characterization. FIG. 5 displays the pictures of M1 and M2 microgels synthesized with different amount of DA. As can been observed, in both cases spherical and monodisperse microgel particles are obtained regardless of the amount of DA. In addition, it seems that increasing the concentration of DA (M2), the edge of the microgel particles is better defined. This could be because being higher the cross-linking density, less dangling chains are.

### 4. Film Formation: Surface Characterization by AFM

Films composed of multilayer of self-assembled microgel were formed following the method described by Boularas et al. (M. Boularas, S. Radji, E. Gombart, J.-F. Tranchat, V. Alard, L. Billon, Materials & Design, 2018, 147, 19-27).

Briefly, 30 mL of purified aqueous microgel dispersion was introduced into inert plastic mold and dried for 48 h at 35° C. (±3° C.) at atmospheric pressure.

Images by Atomic Force Microscopy (AFM) of self-assembled microgel films were recorded on a Multi mode 8 (BrukerNano) using silicon-nitride cantilevers from Bruker-probe (ScanAsyst-Air). The self-assembled microgel films were deposited on wafer silicon substrate prior to be observed by AFM surface mapping.

FIG. 6 shows the AFM micrographs at the surface of the self-assembled M2 purified microgel film. Its surface evidences high ordering of the spherical and monodisperse microgel particles with particle diameters of ˜200 nm (FIG. 6b-6c). Moreover, this area presents blue colored properties. However, the high ordering and colored properties are not observed along whole the film (FIG. 6a). It is important to point out that the film was formed without the need of specific techniques of casting. Maybe, in order to obtain high ordering along the film, a control casting should be used.

Previously the ability of oligo(ethylene glycol)-based self-assembled films to swell maintaining their integrity in aqueous solution was demonstrated.

Films formed with catechol-based microgels were immersed in aqueous solution for 24 hours. Surprisingly, the film formed with M1 microgel particles was re-dispersed. It seems that the attractive forces between the pendant oligo(ethylene glycol) chains at the outer shell of the microgels are not enough to balance the repulsive forces. In the case of the film formed with M2 microgel, the film was swollen maintaining its integrity.

### Example 2

### Incorporation of Poly(3,4-Ethylenedioxythiophene) (PEDOT) Shell Onto Microgels Synthesized

The introduction of poly(3,4-ethylenedioxythiophene) shell onto microgels synthesized using as doping agent the catechol groups of the cross-linker was carried out as follows. In brief, certain amount of microgel at 1% of solid content was placed in a round bottom flask. Subsequently, different amounts of 3,4-ethylenedioxythiophene (EDOT) dissolved in ethanol (from 10 to 25 wt % with respect to microgel) were added and the reaction mixture was stirred at room temperature for few minutes. Then, ammonium persulfate (APS) dissolved in Milli-Q water, maintaining n(APS)/n(EDOT)=1, was added. The polymerization was allowed to continue with stirring until the color was changed to “dark green”.

In addition, a reference core-shell microgel was synthesized adding poly(styrene sulfonate) (PSS) as external doping agent and with 25 wt % of PEDOT with respect to microgel. In that case, conventional microgel synthesized, called as bare microgel (BM), with 2 mol % of oligo(ethylene glycol) diacrylate (OEGDA) was used as seed.

Firstly, the reactions were carried out at room temperature. However, the time needed to complete the synthesis was longer than 1 week. Therefore, in order to increase the reaction kinetics the temperature was increased up to 40° C. and the reaction time was decreased until 4-5 days. In addition, it was observed that decreasing the amount of catechol groups in the microgel particles (MI) the time needed to complete the synthesis was reduced from 4-5 days to 1 day. This suggests that catechol groups were more accessible for EDOT molecules and this could be because catechol groups are mainly located in the surface of the microgel particles.

The recipes and experimental conditions for the synthesis of conductive microgel particles are shown in Table 4. In the first column, the nomenclature of each reaction is shown. The number following PEDOT indicates the weight % of EDOT used with respect to microgel.

TABLE 4
Recipes used to produce conductive microgels

| | Seed | EDOT |
|---|---|---|
| Conductive microgel | (wt %) | (wt % microgel) |
| M1-PEDOT10 | 1 | 10 |
| M1-PEDOT25 | 1 | 25 |
| M2-PEDOT25 | 1 | 25 |
| *BM-PEDOT PSS25 | 1 | 25 |

*Microgel synthesized with conventional cross-linker (OEGDA) and PSS as dopant
Variables: reaction temperature (room temperature or 40° C.)

### 1. Colloidal Characterization

### 1.1. Direct Observation by AFM

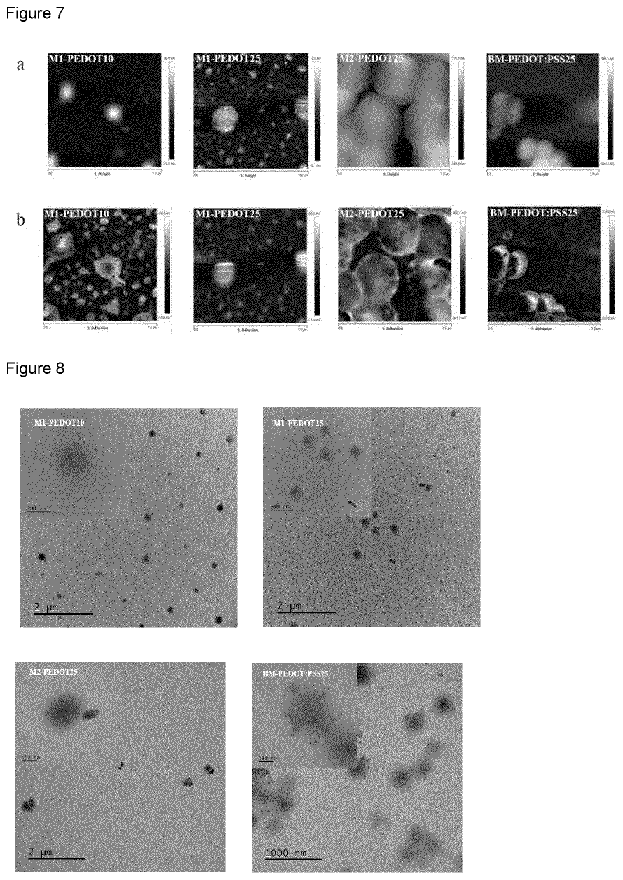

Images by Atomic Force Microscopy (AFM) of microgel particles at dried state were recorded on a Multi mode 8 (BrukerNano). For that, a droplet of microgel dispersion (10⁻² wt %) was deposited on a clean wafer silicon and left to dry under ambient conditions prior to AFM surface mapping of the surface on air. In FIG. 7 the images in height and adhesion mode of different conductive microgels synthesized are shown. In all the cases, after the addition of the conductive shell, the shape of microgel particles is better defined presenting a homogeneous size. As can be observed, in the case of conductive microgels synthesized using the slightly cross-linked microgel (M1) as seed, it seems a homogeneous distribution of PEDOT dots onto the surface of particles (see adhesion mode images). In addition, increasing the amount of PEDOT added, the surface of particles looks better covered by PEDOT dots.

On the other hand, in the case of using highly cross-linked microgel as seed, some dots are observed in the surface of microgel particles but the surface is not completely covered. This could be because some catechol groups are located into microgel particles and part of the EDOT added has polymerized into microgel particles instead of at the surface. Similar images are obtained in the case of reference conductive microgel synthesized using PSS as dopant, i.e. some dots are observed in the surface of the particles but they are not completely covered. It seems that catechol groups are better dopant than PSS ones. However, in order to corroborate this and to obtain images with better resolution, electron microscopy techniques should be used to analyze microgel synthesized.

### 1.2. Direct Observation by TEM

With the aim of corroborating the above hypothesis, conductive microgel samples were sent to Bordeaux Imaging Center to be analyzed by transmission electron microscopy (TEM). In FIG. 8 are shown the images of conductive microgels synthesized using as seed microgels synthesized with different DA concentration and with different EDOT concentrations. As can be seen, in the case of the microgel synthesized using the slightly cross-linked microgel (M1) as seed and lowest PEDOT amount (M1-PEDOT10), a homogeneous distribution of PEDOT dots onto the surface of particles is observed. In addition, no free PEDOT nanoparticles are observed, suggesting that the PEDOT has polymerized only at the surfaces of the microgel particles. However, increasing the concentration of PEDOT (M1-PEDOT25), free PEDOT nanoparticles are observed in the medium and not at the surface of microgel particles. This could be because the amount of EDOT added was too high for the amount of dopant presented in the microgel particles. With the aim of having a complete vision of the microgel particles. STEM-EDS analysis will be carried out. Like this, a map, in terms of concentration, of PEDOT will be obtained, being able to define better the distribution of PEDOT onto/into microgel particles.

On the other hand, in the case of using highly cross-linked microgel as seed, some dots are observed in the surface of microgel particles but the surface is not completely covered; only half of the surface seems to be covered. In addition, the localization of some PEDOT dots into microgel particles is suspected. In the same way, in the case of the conductive microgel synthesized using PSS as dopant, it seems that some PEDOT dots are located into microgel particle. Moreover, in this case, free PEDOT dots are observed in the medium. It seems that the use of catechol groups as dopant is better than the use of conventional PSS dopant in order to attach PEDOT to the surface of microgel particles. With the aim of corroborating the location of PEDOT dots onto/into microgel particles. STEM-EDS analysis will be carried out. Even if more studies are needed to complete the electronic microscopy analyses, it is interesting to point out that the TEM images obtained are in accordance with the AFM ones.

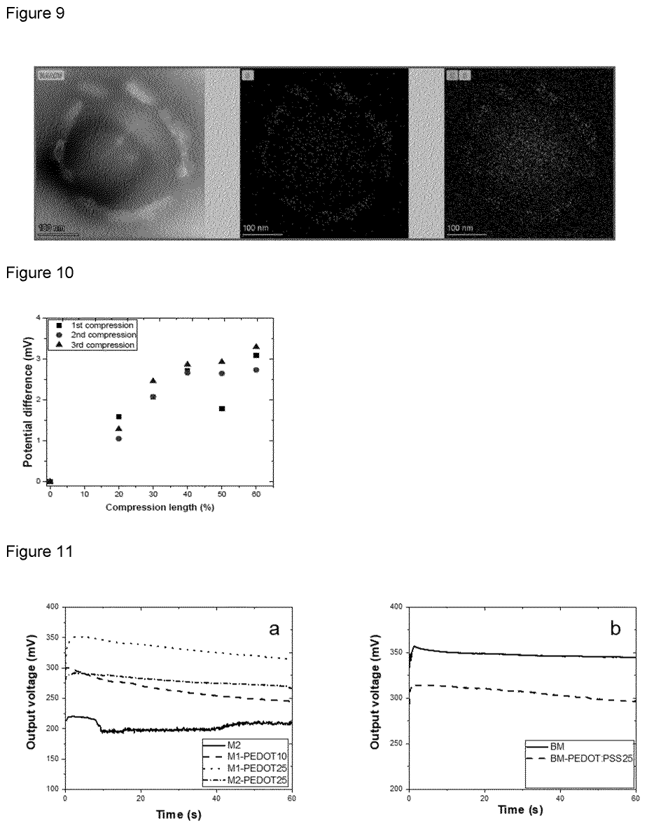

The group of Bordeaux Imaging Center offered the used of more advanced microscopy technique in order to obtain a map with the localization of PEDOT. For that, M2-PEDOT25 sample was analyzed by STEM-EDS and as can be seen in FIG. 9, a ring of PEDOT is observed at the surface of the microgel particle. With this technique, the presence of PEDOT at the surface of microgel particles has been confirmed. In addition, the suitability to catechol groups to efficiently adhere PEDOT to hydrophilic microgel particles has been confirmed.

### 1.3. Direct Observation by MEB-EDS

Conductive self-assembled microgel films were sent to Bordeaux Imaging Center to be analyzed by Scanning Electron Microscopy with Energy Dispersive Spectroscopy (SEM-EDS). In FIG. 12 are shown the images of conductive films with different PEDOT concentrations. As can be seen, a continuous PEDOT network is already obtained for 10 wt % of PEDOT. This means that percolation threshold, which is defined as the point above which enough conductive component has been included to enable the passage of charges and hence confer conductivity properties to the material, is obtained with only 10 wt % of PEDOT. This could be the reason of obtaining similar conductivity values using higher amounts of PEDOT.

### 1.4. Electrophoretic Mobility Measurements

Electrophoretic mobility measurements were carried out by Electrophoretic Light Scattering (ELS), using a Zetasizer Nano ZS instrument (Malvern Instruments). Microgel dispersions were diluted at a concentration of 0.05 wt % using buffered solutions. Each sample was subjected to five measurements at 25° C., without delay between them.

In Table 5, electrophoretic mobility values obtained for different microgels synthesized are shown. The values obtained are in accordance with that observed in AFM images. In the case of conductive microgels synthesized using slightly cross-linked seed (MI-PEDOT10 and MI-PEDOT25), the electrophoretic mobility values are more positive than that obtained for bare seed (MI) meaning lower amount of ionized carboxylic groups at the surface due to the screening of them with PEDOT. By contrast, in the case of conductive microgel synthesized with highly cross-linked seed (M2-PEDOT25), even the amount of PEDOT added was higher, similar electrophoretic mobility value to bare seed was obtained. As it was observed in AFM images, the surface of microgel particles was not completely covered by PEDOT maybe because some PEDOT was polymerized into the microgel particles and therefore, the amount of charges at the surface was maintained constant. In the case of the conductive microgel synthesized using PSS as dopant, more negative values were obtained after the addition of PEDOT due to the deprotonated sulfonate groups located at the surface of the microgel. As it was observed by AFM, some PEDOT dots are observed at the surface of particles being the responsive of the more negative values of electrophoretic mobility obtained.

TABLE 5
Electrophoretic mobility values of microgel synthesized

| | Electrophoretic mobility (m²/Vs * 10⁻⁸) |
|---|---|
| M1 | −1.55 ± 0.06 |
| M2 | −2.04 ± 0.09 |
| M1-PEDOT10 | −0.52 ± 0.03 |
| M1-PEDOT25 | −0.66 ± 0.03 |
| M2-PEDOT25 | −1.91 ± 0.17 |
| BM-PEDOT:PSS25 | −3.38 ± 0.32 |
| BM | −1.65 ± 0.02 |

### 2. Film Characterization

### 2.1. Conductivity Measurements

The conductivity measurements were carried out following 4-wire methodology. For that, films were formed following the procedure described above and the conductivity was calculated considering their thickness. However, when the films were removed from the molds and compressed between the wires, sometimes they broke during the manipulation due to their softness and the measurements were not reliable. Therefore, it was decided to measure directly the resistance of the films without removing them from the silicone molds using electrodes.

Then, conductivity values were calculated as follows:

$$σ = \frac{L}{R * 1000 * e * l}$$

where L and l represents the lengths of the film (cm), longest and shortest sides, respectively, R is the resistance (kΩ) and e is the thickness (cm) of the film.

In Table 6, the conductivity values of the microgel synthesized are presented. As can be seen, the lowest value was obtained in the case of using PSS as dopant. It seems that catechol groups are better dopant for PEDOT polymerization than the usually used PSS. In the case of conductive microgels synthesized using catechol-based seed particles, similar values were obtained independently of the amount of PEDOT. In the case of highly cross-linked seed, higher amount of PEDOT was added but surprisingly similar conductivity than that obtained for the slightly cross-linked microgel with low amount of PEDOT was observed. The reason could be related with the location of PEDOT incorporated. In the case of using slightly cross-linked microgel as seed, PEDOT polymerized mainly in the surface of the microgels. On the other hand, in the case of the highly cross-linked microgel, due to the distribution of catechol groups into particles, some PEDOT was polymerized into the surface but also into microgel particles (see FIG. 6). Therefore, in both cases, the PEDOT polymerized at the surface could be similar leading to similar conductivity values.

TABLE 6
Conductivity values of microgel synthesized

| | Conductivity values (S/cm) |
|---|---|
| M1-PEDOT10 | 2.3 * 10⁻³ |
| M2-PEDOT25 | 2.5 * 10⁻³ |
| BM-PEDOT:PSS25 | 4.9 * 10⁻⁴ |

### 2.2. Mechanoelectrical Transduction of the Microgel Self-Assembled Films

The characterization of mechanoelectrical properties was carried out on home-made setup, by compressing films between two electrodes of Indium Tin Oxide (ITO) coated glass slides (surface resistivity of 12 (2) using a Rheometer in plate-plate geometry connected in an open circuit voltage to detect further electrical potential. To simulate touch-sensitive application on the films, the program proposed by Boularas et al. was followed (M. Boularas, Synthèse de microgels hybrides, biocompatibles et stimulables pour des applications cosmétiques. PHD Thesis, University of Pau et des Pays de l'Adour, 2015).

In brief, the program was composed by a long relaxation time (20 s) at zero displacement equivalent to the hydrated film thickness and short period of compression of the film (2 s) at different displacements from 0 to 60%. Each step of compression/relaxation was repeated three times in a row. The variation of electrical potential (E) was recorded as a function of magnitude of compression and output voltage between the two electrodes was recorded by using LabVIEW software.

In FIG. 10, the output voltage generated under repeated compression of the M2 microgel self-assembled film is shown. An increase of the output voltage with the magnitude of compression is observed, as expected. In addition, constant output voltage values are obtained upon successive compression cycles. It seems that the relaxation period between cach compression was sufficient to complete relaxation of the hydrated films.

It is important to notice that it was not possible to complete the whole measurement process for all the films due to their softness. In some cases, the output was not observed until 30-40% of compression and in other cases, at high compression lengths (50-60%) some of them were broken. Therefore, in the further part, the first compression of 30% displacement cycle was used to accurately characterize the effect of compression on the generated electrical potential according to the type of film (see Table 7).

The maximum output voltage was obtained in the case of the microgel synthesized with slightly cross-linked seed and low amount of PEDOT (MI-PEDOT10). As it was observed by AFM, in that case the surface of the particles was well covered by PEDOT. In the case of the film formed with M2-PEDOT25 (highly cross-linked seed and high amount of microgel), a lower output voltage than bare seed (M2) was observed.

As it was discussed above, part of the PEDOT could be polymerized into microgel particles and this could hinder ions mobility screening ionized carboxylic groups. Finally, the film formed using conductive microgel synthesized with conventional dopant (BM-PEDOT:PSS25) presented similar output voltage to that presented by bare microgel (BM). This means that there was no an enhancement of mechanoelectrical properties after the addition of a conductive polymer.

TABLE 7
Output voltage (mV) generated at 30% of compression
for different microgel self-assembled films

| | Output voltage (mV) |
|---|---|
| BM | 2.3 |
| M2 | 2.1 |
| M1-PEDOT10 | 3.3 |
| M2-PEDOT25 | 1.6 |
| BM-PEDOT:PSS25 | 2.1 |

With the aim of mimicking skincare applications, the output voltage was measured compressing the films directly by the finger. For that, some modifications were carried out in the home-made setup. The ITO slide was covered with adhesive tape saving a small part for film deposition. Then, copper tape was fix to a finger in order to use it as a second electrode. Finally, the films were compressed smoothly with the finger during few seconds and the output voltage was recorded by using LabVIEW software.

In Table 8, the output voltage values after finger compression obtained are shown. As can be seen, in all the cases, the values are 10-15 times higher than those obtained by compressing between two ITO slides. This could be because the contact between the finger and the film is much better than that between the ITO slide and the film. In addition, from the point of view of skincare applications, reversible pore induction in cell membranes and lipid bilayer membranes has been observed at 150-250 mV (several seconds)(H. Inada, A.-H. Ghanem, W. I. Higure, Pharm. Res., 1994, 11, 687-697).

Therefore, the films formed should be able to create new pores in the skin enhancing the penetration of the active molecules.

TABLE 8
Output voltage (mV) generated after finger compression
for different microgel self-assembled films.

| | Output voltage (mV) |
|---|---|
| BM | 235.2 ± 5.9 |
| M2 | 193.8 ± 19.8 |
| M1-PEDOT10 | 213.4 ± 9.3 |
| M2-PEDOT25 | 148.7 ± 21.1 |
| BM-PEDOT:PSS25 | 281.7 ± 6.0 |

However, in order to compare the values obtained among them, a reference of the compression made with the finger is needed. As explained above, the output voltage depends on the compression length and therefore, the same pressure of compression should be applied with the finger in order to compare the values among them. In this regard, the setup was modified to measure the force generated after the compression with the finger together with the output voltage. For that, ITO slide covered with adhesive tape, saving a small part for film deposition, was deposited onto a force sensor. Then, copper tape was fix to a finger in order to use it as a second electrode.

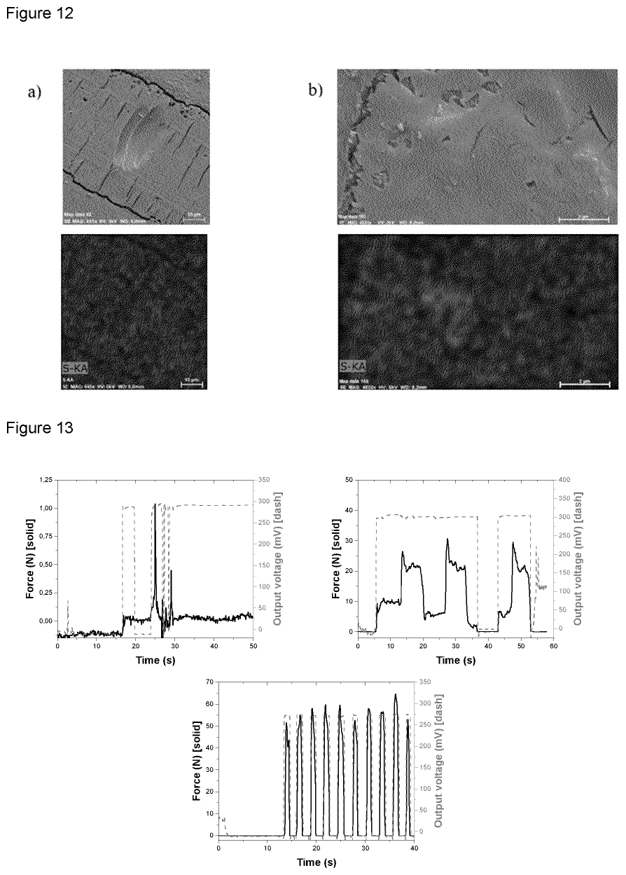

In FIG. 11, the obtained output voltage values after applying a fixed force by finger are shown. The applied force was fixed to 10-15 N being this value range similar to that applied with a finger during the common application of a cream.

As can be observed, the output voltage is maintained constant at least during 1 minute, in all the cases. Moreover, using catechol groups as seed, the obtained output voltages after PEDOT incorporation are higher, confirming the efficiency of catechol groups as dopant. By contrast, in the case of using the PSS as dopant, the obtained output voltage after PEDOT incorporation is lower (FIG. 11b). It seems, that catechol groups are more efficient as dopant agent than PSS in the case of incorporating PEDOT shell onto microgel particles. Finally, increasing the PEDOT concentration from 10 to 25 wt %, higher output voltage is obtained, as expected (FIG. 11a).

### Enhancing the Output Voltage Generated

### Force Applied

The effect of different parameters on mechanoelectrical transduction properties of slightly cross-linked microgel particles with 10 wt % PEDOT were studied generating a mechanical stress by compression of the hydrated films applying a force by finger. The first parameter analyzed was the force applied. For that, 3 different forces were applied: 25 N, 55 N and the force applied just placing the electrode onto the film (less than 1 N). As can be seen in FIG. 13. The force applied just putting the electrode onto the film is enough to generate the maximum output voltage. Therefore, it can be considered that the force applied has no effect on the output voltage generated.

### Arca

With the aim of enhancing the output voltage generated, different parameters of the films (arca and thickness) formed using slightly cross-linked microgel particles with 10 wt % PEDOT were modified.

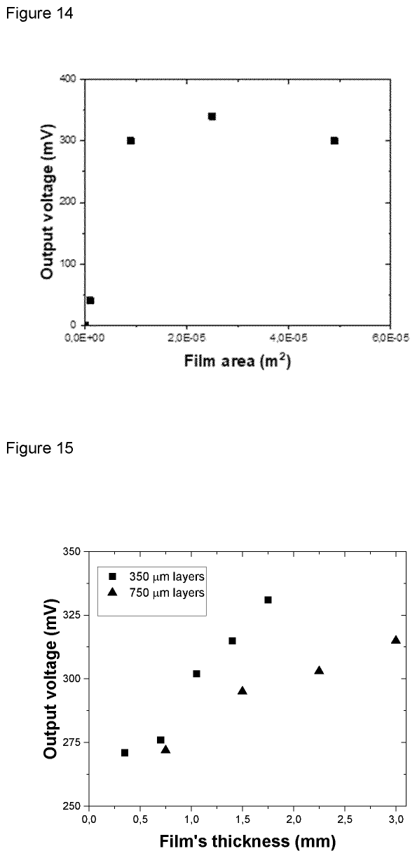

The applied force was fixed to 10-15 N being this value range similar to that applied with a finger during the common application of a cream. On the one hand, it was observed that the increment of the films arca had almost no effect on the electrical potential, i.e. maximum output voltage was obtained with really small films area (see FIG. 14). However, for smaller surfaces than 1E-05 m2. it was possible to control the output voltage generated.

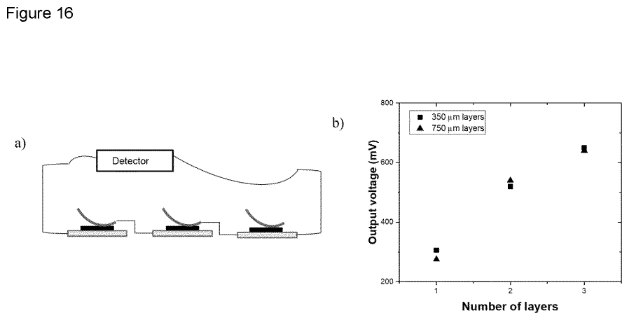

On the other hand, the thickness of the film was increased through the deposition of different layers of 350 and 750 μm onto the electrode. As can be observed in FIG. 15, a slight increase of the output voltage was observed after the deposition of several layers, in both cases. In fact, small increment of ˜50 mV was only observed after tripled the thickness of the film for both types of films. The reason could be the non-complete contact between different layers of film. In addition, even for both films a linear increase of the output voltage was observed, the increment in the case of the thicker film was lower. This could be due to the higher defects inside thicker films. With the aim of increasing the electrical potential generated, the connection of multiple small film units (9×10−6 m2) together was studied. For that, the different film units with different thickness (350 or 750 μm) were connected in series as can be seen in the schematic illustration of FIG. 16a.

An increase in the output voltage was observed increasing the number of electrodes placed in series obtaining a huge value of ˜700 mV for both films (FIGS. 16b). However, no effect of the film thickness was observed, at least in the range studied.

Advantageously, it has been demonstrated that using small piece of self-assembled microgel film (9×10−6 m2), a high electrical potential can be generated being possible to amplify it combining the appropriate number of films in series.

1. A composition comprising an oligo(ethylene glycol)-based polymers microgel crosslinked with a catechol cross-linker, wherein said oligo(ethylene glycol)-based polymer microgel comprises microgel particles, and wherein the microgel particles have a shell of conductive polymer.

2. The composition according to claim 1, wherein the catechol cross-linker comprises an acrylamide or methacrylamide group.

3. The composition according to claim 1, wherein the catechol cross-linker is dopamine-acrylamide or dopamine methacrylamide.

4. The composition according to claim 1, wherein the conductive polymer is selected from the group consisting of poly(3,4-ethylenedioxythiophene) (PEDOT), PEDOT derivatives and poly(3-hexylthiophen) (P3HT).

5. The composition according to claim 1, wherein the conductive shell is a discontinuous conductive shell.

6. The composition according to claim 1, wherein the microgel is obtainable by aqueous phase precipitation polymerization of one or more of the following monomers:
di(ethylene glycol) methyl ether methacrylate (MeO₂MA);
an oligo(ethylene glycol) methyl ether methacrylate (M(EO)ₙMA), n being an integer ranging from 3 to 12 and
a monomer of formula CR₁R₂═CR₃R₄ in which R₁, R₂, R₃ and R₄ represent a hydrogen, a halogen or a hydrocarbon group, at least one of the four groups comprising a —COOH or —COO⁻M⁺ group, M⁺ representing a cation, cross-linked with a catechol cross-linker.

7. The composition according to claim 1, wherein the microgel is obtained via aqueous phase precipitation polymerization of monomers cross-linked with the catechol cross-linker, said monomers being di(ethylene glycol) methyl ether methacrylate (MeO₂MA), oligo(ethylene glycol) methyl ether methacrylate (OEGMA) and methacrylic acid (MAA).

8. A process of preparing a composition according to claim 1, said process comprising the steps of:
preparing a microgel via precipitation polymerization of monomers, wherein said monomers are crosslinked with a catechol cross-linker, wherein said microgel comprises particles and wherein the monomers are selected from the group consisting of:
di(ethylene glycol) methyl ether methacrylate (MeO₂MA);
an oligo(ethylene glycol) methyl ether methacrylate (M(EO)ₙMA), n being an integer ranging from 3 to 12, preferably ranging from 8 to 10, and a monomer of formula CR₁R₂═CR₃R₄ in which R₁, R₂, R₃ and R₄ represent a hydrogen, a halogen or a hydrocarbon group, at least one of the four groups comprising a —COOH or —COO⁻M⁺ group, M⁺ representing a cation, and
preparing a conductive shell via polymerization on microgel particles.

9. A Self-Assembled Microgel Film wherein said Self-Assembled Microgel Films is obtained by solvent evaporation of a composition according to claim 1.

10. A process of obtaining a Self-Assembled Microgel Films comprising a step of applying on keratin materials a composition according to claim 1.

11. A cosmetic product comprising a composition according to claim 1 and at least one cosmetic agent, wherein the microparticles comprise the cosmetic agent.

12. A make-up or a skin care method comprising applying on keratinous materials a cosmetic product according to claim 11 and applying a compression on said product.

13. A therapeutic product comprising a composition according to claim 1 and a therapeutic agent, wherein the microparticles comprise the therapeutic agent.

14. (canceled)

15. A series of films obtained by drying or evaporating solvent of from a compositions according to claim 1, or from a Self-Assembled Microgel Film made with the composition, wherein each film or each Self-Assembled Microgel Film in the series of films, is connected, respectively, to another film or Self-Assembled Microgel Film.

16. A method for delivering a therapeutic agent to a keratinous material comprising:
i) applying to the keratinous material a therapeutic product of claim 13, a microgel of the therapeutic product being in the form of an aqueous dispersion or series of aqueous dispersions;
ii) obtaining at least one self-assembled microgel film by solvent evaporation, wherein the therapeutic agent is delivered to the keratinous material from the at least one self-assembled microgel film.

17. The method of claim 16, wherein step ii) is repeated a plurality of times to obtain a plurality of layers of self-assembled microgel films, and wherein the method further comprises
iii) connecting the plurality of layers by applying compression thereto.

18. The method of claim 6, wherein n ranges from 8 to 10.

19. The process of claim 8, wherein n ranges from 8 to 10.
